(19) United States
(12) Patent Application Publication
(10) Pub. No.: US 2024/0247230 A1
(43) Pub. Date: Jul. 25, 2024

(54) APPLICATION OF GENETICALLY MODIFIED OLIGODENDROCYTE PROGENITOR CELLS IN MULTIPLE SCLEROSIS

(71) Applicant: ALLIFE MEDICINE (BEIJING) LIMITED, Beijing (CN)

(72) Inventors: Yuchun GU, Beijing (CN); Lida WU, Beijing (CN)

(21) Appl. No.: 18/592,249

(22) Filed: Feb. 29, 2024

Related U.S. Application Data
(63) Continuation of application No. PCT/CN2022/116751, filed on Sep. 2, 2022.

(30) Foreign Application Priority Data
Sep. 3, 2021 (CN) 202111033274.7

(51) Int. Cl. C12N 5/079; A61K 35/30; A61K 38/00; A61P 25/28; C07K 14/52; C07K 14/54; C12N 5/00; C12N 15/86

(52) U.S. Cl. CPC C12N 5/0622; A61K 35/30; A61P 25/28; C07K 14/521; C07K 14/5403; C07K 14/5428; C12N 5/0018; C12N 15/86; A61K 38/00; C12N 2500/02; C12N 2500/32; C12N 2500/38; C12N 2500/44; C12N 2501/01; C12N 2501/12; C12N 2501/135; C12N 2501/33; C12N 2501/999; C12N 2506/45; C12N 2510/00; C12N 2523/00; C12N 2740/15043

(57) ABSTRACT

Provided are a genetically modified oligodendrocyte progenitor cell, a preparation method therefor and a use thereof. Also provided is a method capable of simultaneously repairing myelin, promoting myelin production, and reducing inflammatory responses and autoimmune damage; the method comprises a genetically engineered oligodendrocyte progenitor cell achieving direct repair of a myelin sheath by means of transplantation of the genetically modified oligodendrocyte progenitor cell, which can alleviate an inflammatory response of the nerve and improve nerve function. This has very good application prospects in the clinical treatment of multiple sclerosis.

### SEQUENCE LISTING

The instant application contains a sequence listing which has been submitted electronically in the XML file format and is herein incorporated by reference in its entirety. (Filename:“DOP2422751001PUS.xml”; Date created: Feb. 8, 2024; File size: 13,637 Bytes).

### TECHNICAL FIELD

The present invention relates to the field of biomedicine, and in particular, to an application of a genetically modified oligodendrocyte progenitor cell in multiple sclerosis, and more particularly, to a genetically modified oligodendrocyte progenitor cell, preparation methods and using thereof.

### BACKGROUND

Multiple sclerosis (MS) is an immune mediated chronic demyelinating diseases which is the most common in central nervous system (CNS), mainly occurs in young patients, and is the most common cause of disability of young people second to injury. There are multiple inflammatory demyelination plaques in the central white matter in the acute activity period of the disease. The old lesions formed calcified plaques due to the proliferation of glia fiber. The disease is characterized by multiple lesions, remission and relapse in the courses of disease, and most common in optic nerves, spinal cord and brain stem, mainly occurs in adolescent, middle-aged people and affects more women than men. The middle age of the disease is 29. The number of female patients is 3 times as that of male patients. The disease cannot be radically cured, has a high rate of disability, and seriously affects health and life quality. The first MS map was issued by the MS International Federation (MSIF) and the World Health Organization (WHO), wherein the data show that occurrence areas of MS are more concentrated in Europe and eastern Mediterranean, the morbidity is 100-200/100 thousand. The morbidity in Asian is low, and the morbidity in China is 0-5/100 thousand and with a rising trend in recent years (Milo R, Kahana E. Multiple sclerosis: geoepidemiology, genetics and the environment[J]. Autoimmun Rev, 2010, 9(5): A387-A394.).

At present, the main treatment of MS is divided into acute stage treatment and remission stage treatment. The treatment in the remission stage is named Disease Modification Therapy (DMT). The drugs mainly applied comprise β-interferon, teriflunomide, Natalizumab, mitoxantrone and the like. As MS is caused by the damage of nerve myelin by autoimmune cells, the measures in therapy mainly include inhibiting immune cells, reducing immune cells from penetrating the blood-brain barrier, and promoting differentiation of oligodendrocyte progenitor cells (OPC) to recreate myelin (Wingerchuk D M, Carter J L. Multiple sclerosis: current and emerging disease-modifying therapies and treatment strategies[C]//Mayo Clinic Proceedings. Elsevier, 2014, 89(2):225-240.; Gholamzad M, Ebtekar M, Ardestani M S, et al. A comprehensive review on the treatment approaches of multiple sclerosis: currently and in the future[J]. Inflammation Research, 2019, 68(1):25-38.).

As the high recurrence rate, difficulty of radical treatment and high disability rate of MS patients, current research scholars have done relevant exploration of stem cell therapy for MS clinically, including transplantation therapies of stem cells related to bone marrow-derived mesenchymal stem cells (MSC), adipose-derived MSC, umbilical cord-derived MSC, neural stem cell (NSC), MSC-differentiated neural precursors (NPs), haematopoietic stem cells (NSC), and the like. Although to some degree, the use of stem cells promotes the generation of myelin, however, inflammatory response and autoimmune injury cannot be reduced, and therefore, the overall effect of symptom relieving by relevant stem cell transplantation treatments for MS is not obvious in current researches. In addition, OPC cannot be separated from human body, although OPC can repair the myelin, is currently still impossible to be carried forward clinically.

The present invention aims to provide a method capable of repairing myelin, promoting the generation of myelin, and reducing inflammatory response and autoimmune injury simultaneously. The present invention can repair myelin directly through transplantation of genetically modified OPC, meanwhile, protective layers are locally formed around the nerves by secretion of anti-inflammatory factors IL-27, IL-10 and the like, so that the inflammatory response of nerves is reduced. Meanwhile, T-reg cells, M2 cells recruited by CXCL11 and so on, are enriched around the nerve myelin to protect the myelin from damage. And the neural function is improved by maintaining the signal adjustment of astrocyte, microglia and nerves through the secretion of IL-3.

### SUMMARY

In view of the above, in order to overcome the above-mentioned technical defects in the present field, the purpose of the present invention is to provide a method capable of repairing myelin, promoting the generation of myelin, reducing inflammatory response and autoimmune injury simultaneously, the method comprising the genetically modified oligodendrocyte progenitor cell.

The above object of the present invention is achieved by the following technical solutions:

In a first aspect, the present invention provides a construct for genetically modifying an induced pluripotent stem cell to obtain a genetically modified oligodendrocyte progenitor cell.

Further, the construct comprises nucleotides encoding anti-inflammatory cytokines and/or nucleotides encoding chemokines;

Preferably, the anti-inflammatory cytokines include: IL-10, IL-27, IL-3, IL-2, IL-4, IL-6, IL-10, IL-11, IL-12, IL-13, IL-16, IL-18, IL-22, IL-27, IL-35, IL-37, IL-38, IL-1Ra, TGF-β;

More preferably, the anti-inflammatory cytokines are IL-10, IL-27, or IL-3;

Preferably, the chemokines include: a CXC chemokine subgroup, a CC chemokine subgroup, an XC chemokine subgroup, and a CX3C chemokine subgroup;

More preferably, the CXC chemokine subgroup includes: CXCL11, CXCL1, CXCL2, CXCL3, CXCL4, CXCL5, CXCL6, CXCL7, CXCL8, CXCL9, CXCL10, CXCL12, CXCL13, CXCL14, CXCL15, CXCL16, CXCL17.

More preferably, the CC chemokine subgroup includes: CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28.

More preferably, the XC chemokine subgroup includes: XCL1, XCL2;

More preferably, the CX3C chemokine subgroup includes CX3CL1;

Most preferably, the chemokines are CXCL11.

In a second aspect, the present invention provides a vector.

Further, a vector comprises the construct according to the first aspect of the present invention.

Preferably, the vector comprises the nucleotide encoding IL-10, the nucleotide encoding IL-27, the nucleotide encoding IL-3, and/or the nucleotide encoding CXCL11;

Preferably, the vector includes: the DNA vector, the viral vector;

Most preferably, the DNA vector includes: the DNA plasmid vector, the liposome that binds to the DNA plasmid, the molecular conjugate that binds to the DNA plasmid, and the polymer that binds to the DNA plasmid;

Most preferably, the viral vector includes: the adenovirus vector, the adeno-associated virus vector, the lentiviral vector, the retroviral vector, the herpes simplex virus vector, the baculovirus vector, the Sendai virus vector, the poxvirus vector, and the geminivirus vector.

In a third aspect, the present invention provides a genetically modified induced pluripotent stem cell.

Further, the genetically modified induced pluripotent stem cell expresses IL-10, IL-27, IL-3, and/or CXCL11.

Preferably, the genetically modified induced pluripotent stem cell overexpresses IL-10, IL-27, IL-3, and/or CXCL11;

More preferably, the genetically modified induced pluripotent stem cell overexpresses IL-10, IL-27, IL-3, and CXCL11;

Preferably, the genetically modified induced pluripotent stem cell comprises the construct according to the first aspect of the present invention, and/or the vector according to the second aspect of the present invention.

In a fourth aspect, the present invention provides a genetically modified oligodendrocyte progenitor cell.

Further, the genetically modified oligodendrocyte progenitor cell expresses IL-10, IL-27, IL-3, and/or CXCL11;

Preferably, the genetically modified oligodendrocyte progenitor cell overexpresses IL-10, IL-27, IL-3, and/or CXCL11;

More preferably, the genetically modified oligodendrocyte progenitor cell overexpresses IL-10, IL-27, IL-3, and CXCL11

In a fifth aspect, the present invention provides a method for preparing the genetically modified induced pluripotent stem cell according to the third aspect of the present invention.

Further, the method comprises: delivering the vector according to the second aspect of the present invention to an induced pluripotent stem cell;

Preferably, the delivering is achieved by introducing the vector according to the second aspect of the present invention into an induced pluripotent stem cell;

More preferably, the method of said introducing includes microinjection, electroporation, DEAE-glucan mediated transfection, TALEN method, ZFN method, non-viral vector mediated transfection, viral vector mediated transfection, transposon technique, CRISPR-Cas 9 technique;

Most preferably, the non-viral vector mediated transfection includes liposome transfection, calcium phosphate transfection, and chitosan transfection;

Most preferably, the viral vector mediated transfection includes lentivirus infection, retroviral infection, adenovirus infection, adeno-associated virus infection.

In a sixth aspect, the present invention provides a method for preparing the genetically modified oligodendrocyte progenitor cell according to the fourth aspect of the present invention.

Further, the method comprises: induction differentiation of the genetically modified induced pluripotent stem cell according to the third aspect of the present invention to obtain the genetically modified oligodendrocyte progenitor cell;

Preferably, the induction differentiation comprises the following steps:

(1) first stage induction differentiation: culturing the genetically modified induced pluripotent stem cell according to the third aspect of the present invention with basal medium added with GlutaMAX-I, 2-Mercaptoethanol, SB431542, LDN193189, vitamin A acid, and insulin;

(2) second stage induction differentiation: culturing the cell obtained in step (1) with the basal medium added with GlutaMAX-I, 2-Mercaptoethanol, N2 supplement, SAG, and vitamin A acid;

(3) third stage induction differentiation: culturing the cell obtained in step (2) with the basal medium added with GlutaMAX-I, 2-Mercaptoethanol, N2 supplement, B27 supplement, SAG, vitamin A acid and insulin;

(4) fourth stage induction differentiation: culturing the cell obtained in step (3) with the basal medium added with GlutaMAX-I, 2-Mercaptoethanol, N2 supplement, B27 supplement, PDGF-AA, IGF-1, HGF, NT3, T3, Biotin, cAMP, and insulin to obtain the genetically modified oligodendrocyte progenitor cell;

More preferably, the first stage induction differentiation is for 5-9 days in total;

More preferably, the second stage induction differentiation is for 2-6 days in total;

More preferably, the third stage induction differentiation is for 6-10 days in total;

More preferably, the fourth stage induction differentiation is for 9-13 days in total;

Most preferably, the first stage induction differentiation is for 7 days in total;

Most preferably, the second stage induction differentiation is for 4 days in total;

Most preferably, the third stage induction differentiation is for 8 days in total;

Most preferably, the fourth stage induction differentiation is for 11 days in total;

More preferably, the culture condition is 37° C./5% CO₂;

More preferably, the basal medium is DMEM/F-12 medium.

In a seventh aspect, the present invention provides an induction differentiation agent for induction differentiation of the genetically modified induced pluripotent stem cells according to the third aspect of the present invention to obtain the genetically modified oligodendrocyte progenitor cells.

Further, the induction differentiation agent includes a first stage induction differentiation agent, a second stage induction differentiation agent, a third stage induction differentiation agent, and a fourth stage induction differentiation agent;

Preferably, the first stage induction differentiation agent is consisting of: GlutaMAX-I, 2-Mercaptoethanol, SB431542, LDN193189, vitamin A acid, and insulin;

More preferably, the first stage induction differentiation agent further comprises the non-essential amino acid;

Most preferably, the content of each component in the first stage induction differentiation agent is respectively: 1% of the non-essential amino acid, 1% GlutaMAX-I, 0.1 mM 2-Mercaptoethanol, 10 μM SB431542, 0.25 μM LDN193189, 100 μM of vitamin A acid, and 25 μg/mL of insulin;

Preferably, the second stage induction differentiation agent is consisting of: GlutaMAX-I, Mercaptoethanol, N2 supplement, SAG, vitamin A acid;

More preferably, the second stage induction differentiation agent further comprises the non-essential amino acid;

Most preferably, the content of each component in the second stage induction differentiation agent is respectively: 1% non-essential amino acid, 1% GlutaMAX-I, 0.1 mM 2-Mercaptoethanol, 1% N2 supplement, 1 μM SAG, and 100 μM of vitamin A acid;

Preferably, the third stage induction differentiation agent is consisting of: GlutaMAX-I, 2-Mercaptoethanol, N2 supplement, B27 supplement, SAG, vitamin A acid, and insulin;

More preferably, the third stage induction differentiation agent further comprises the non-essential amino acid.

Most preferably, the content of each component in the third stage induction differentiation agent is respectively: 1% non-essential amino acid, 1% GlutaMAX-I, 0.1 mM 2-Mercaptoethanol, 1% N2 supplement, 2% B27 supplement, 1 μM SAG, 100 μM vitamin A acid, and 25 μg/mL insulin;

Preferably, the fourth stage induction differentiation agent is consisting of: GlutaMAX-I, 2-Mercaptoethanol, N2 supplement, B27 supplement, PDGF-AA, IGF-1, HGF, NT3, T3, Biotin, cAMP, insulin;

More preferably, the fourth stage induction differentiation agent further comprises the non-essential amino acid.

Most preferably, the content of each component in the fourth stage induction differentiation agent is: 1% non-essential amino acid, 1% GlutaMAX-I, 0.1 mM 2-Mercaptoethanol, 1% N2 supplement, 2% B27 supplement, 10 ng/mL PDGF-AA, 10 ng/mL IGF-1, 5 ng/mL HGF, 10 ng/mL NT3, 60 ng/mL T3, 100 ng/mL Biotin, 1 μM cAMP, 25 μg/mL insulin.

In an eighth aspect, the present invention provides a kit for producing the genetically modified induced pluripotent stem cells according to the third aspect of the present invention, and/or the genetically modified oligodendrocyte progenitor cells according to the fourth aspect of the present invention.

Further, the kit includes:
(I) the constructs according to the first aspect of the present invention, and/or
(II) the vectors according to the second aspect of the present invention, and/or
(III) induced pluripotent stem cells, and/or
(IV) one or more culture media;

Preferably, the culture medium is the basal medium added with the induction differentiation agent according to the seventh aspect of the present invention;

More preferably, the basal medium is DMEM/F-12 medium.

In a ninth aspect, the present invention provides a composition.

Further, the composition comprises the construct according to the first aspect of the present invention, and/or the vector according to the second aspect of the present invention, and/or the genetically modified induced pluripotent stem cell according to the third aspect of the present invention, and/or the genetically modified oligodendrocyte progenitor cell according to the fourth aspect of the present invention.

Preferably, the composition includes a pharmaceutical composition;

More preferably, the pharmaceutical composition comprises the genetically modified induced pluripotent stem cell according to the third aspect of the present invention, and/or the genetically modified oligodendrocyte progenitor cell according to the fourth aspect of the present invention;

More preferably, the pharmaceutical composition further comprises pharmaceutically acceptable vectors and/or auxiliary materials.

More preferably, the pharmaceutical composition further comprises one or more therapeutic agents;

Most preferably, the therapeutic agent includes: peptide, cytokine, checkpoint inhibitor, mitogen, growth factor, miRNA, dsRNA, mononuclear blood cell, feeder cell, feeder cell component or replacement factor thereof, antibody, chemotherapeutic agent, and immunomodulatory drug.

Further, the pharmaceutically acceptable vectors and/or auxiliary materials includes, but is not limited to, buffers such as neutral buffered saline, phosphate buffered saline, and the like; carbohydrates such as glucose, mannose, sucrose or glucan, mannitol; proteins; polypeptides or amino acids such as glycine; antioxidants; chelating agents such as EDTA or glutathione; adjuvants (e.g. aluminum hydroxide); and preservatives. The suitable pharmaceutically acceptable vector, diluent or excipient is described in detail in Remington's Pharmaceutical Sciences (19th ed., 1995).

Further, the pharmaceutical composition may also be used in combination with other drugs, and the other drugs include, but are not limited to, Methylprednisolone (MPL), Interferon-β-1a (IFN-β-a), Interferon-β-1b(IFN-β-b), Glatiramer, Mitoxantrone, Natalizumab, Fingolimod, Zeposia (Ozanimod), Teriflunomide, Dimethyl Fumarate (DMF), Alemtuzumab, Ocrevus(Ocrelizumab), Rituximab(Rituxan), Mayzent (Siponimod), Cladribine, Laquinimod, Kesimpta(ofatumumab), Ponvory (Ponesimod), Ampyra (dalfampridine).

Further, dosage forms of the pharmaceutical composition include, but are not limited to, injection, tablet, capsule, aerosol, immune preparation, granule, ointment, pill, oral liquid, inhalant, liniment, tincture, suppository.

Further, the administration route of the pharmaceutical composition includes, but is not limited to, arterial injection, intravenous injection, intramuscular injection, subcutaneous injection, intradermal injection, bone marrow injection, inhalation administration, nasal administration, transdermal administration, intraperitoneal injection, epidural injection, spinal cord injection.

Further, the appropriate administration dosage of the pharmaceutical composition according to the present invention can be given a variety of prescriptions according to factors such as formulation method, administration method, patient's age, weight, gender, disease conditions, diet, administration time, administration route, excretion speed and response sensitivity, and skilled practitioners can generally easily determine the prescription and prescribe administration dosage effective for desired treatment or prevention.

In a tenth aspect, the present invention provides an application in any one of the following aspects:
(1) application of the construct according to the first aspect of the present invention in preparation of a vector;
(2) application of the construct according to the first aspect of the present invention in preparation of a kit for producing the genetically modified induced pluripotent stem cell according to the third aspect of the present invention, and/or the genetically modified oligodendrocyte progenitor cell according to the fourth aspect of the present invention.
(3) application of the vector according to the second aspect of the present invention in preparation of the genetically modified induced pluripotent stem cell according to the third aspect of the present invention, and/or the genetically modified oligodendrocyte progenitor cell according to the fourth aspect of the present invention;
(4) application of the vector according to the second aspect of the present invention in preparation of the drug for treating and/or preventing multiple sclerosis;
(5) application of the vector according to the second aspect of the present invention in preparation of a kit for producing the genetically modified induced pluripotent stem cell according to the third aspect of the present invention, and/or the genetically modified oligodendrocyte progenitor cell according to the fourth aspect of the present invention.
(6) application of the genetically modified induced pluripotent stem cell according to the third aspect of the present invention in preparation of the terminally differentiated cell or the precursor cell thereof;
(7) application of the genetically modified induced pluripotent stem cell according to the third aspect of the present invention in preparation of the genetically modified oligodendrocyte progenitor cell;
(8) application of the genetically modified induced pluripotent stem cell according to the third aspect of the present invention in preparation of the drug for treating and/or preventing multiple sclerosis;
(9) application of the genetically modified oligodendrocyte progenitor cell according to the fourth aspect of the present invention in preparation of a drug for treating and/or preventing multiple sclerosis;
(10) application of the induction differentiation agent according to the seventh aspect of the present invention in preparation of the genetically modified oligodendrocyte progenitor cell;
(11) application of the kit according to the eighth aspect of the present invention in the production of the genetically modified induced pluripotent stem cell and/or the genetically modified oligodendrocyte progenitor cell.
(12) application of the composition according to the ninth aspect of the present invention in preparation of a drug for treating and/or preventing multiple sclerosis;
(13) application of IL-10, IL-27, IL-3 and CXCL11 in preparation of the genetically modified induced pluripotent stem cell for treating and/or preventing multiple sclerosis;
(14) application of IL-10, IL-27, IL-3 and CXCL11 in preparation of the genetically modified oligodendrocyte progenitor cell for treating and/or preventing multiple sclerosis;
(15) application of IL-10, IL-27, IL-3 and CXCL11 in preparation of a drug for treating and/or preventing multiple sclerosis.

The present invention also provides the method of expressing IL-10, IL-27, IL-3, CXCL11 in a subject in need thereof.

Further, the method comprises administering to a subject in need an effective amount of the vectors according to the second aspect of the present invention, and/or the genetically modified induced pluripotent stem cells according to the third aspect of the present invention, and/or the genetically modified oligodendrocyte progenitor cells according to the fourth aspect of the present invention.

The present invention also provides a method of treating and/or preventing multiple sclerosis.

Further, the method comprises administering to a subject in need an effective amount of the genetically modified induced pluripotent stem cells according to the third aspect of the present invention, and/or the genetically modified oligodendrocyte progenitor cells according to the fourth aspect of the present invention, and/or the pharmaceutical composition according to the seventh aspect of the present invention, and/or the composition according to the ninth aspect of the present invention.

Compared with the prior art, the present invention has the following advantages and beneficial effects:

In the prior art, although the generation of myelin is promoted to a certain extent, inflammatory response and autoimmune injury cannot be reduced; The transplantation using the genetically modified oligodendrocyte progenitor cells prepared by present invention can repair the myelin, promote the generation of the myelin, reduce inflammatory response and autoimmune injury simultaneously, thereby a good treatment effect on multiple sclerosis is achieved, the overall improvement effect is remarkable;

In the present invention, direct reparation of myelin can be achieved by transplantation using the genetically modified OPCs; meanwhile, protective layers are locally formed around the nerves by secretion of anti-inflammatory factors of IL-27, IL-10 and the like, so that the inflammatory response of nerves is reduced; meanwhile, T-reg cells, M2 cells recruited by CXCL11 and so on, are enriched around the nerve myelin to protect the myelin from injury. And the neural function is improved by maintaining the signal adjustment of astrocyte, microglia and nerves through the secretion of IL-3; therefore, the present invention has a very good application prospect in the aspect of clinical treatment of multiple sclerosis.

### BRIEF DESCRIPTION OF THE FIGURES

Embodiments of the present invention are described in detail below in conjunction with the figures, in which:

FIG. 1 shows a structural diagram of the TEToff-CXCL-puromycin vector;

FIG. 2 shows a structural diagram of the IL10-T2A-IL27-Zeo;

FIG. 3 shows a structural diagram of the TEToff-IL10-T2A-IL27-Zeo vector;

FIG. 4 shows a structural diagram of the IL3-hygroR;

FIG. 5 shows a structural diagram of the TEToff-IL3-hygroR vector;

FIG. 6 shows the results of the 30th day OPC marker Olig2 (green), Nestin (red) gene identification;

FIG. 7 shows the results of the immunohistochemistry of myelin basic protein antibody (MBP), MBP is green fluorescence, DAPI is blue fluorescence, wherein, image A: control group, image B: CPZ group;

FIG. 8 shows the results of immunohistochemistry of hippocampal region of mouse, MBP is red fluorescence, Olig2 is green fluorescence, DAPI is blue fluorescence, wherein, image A: control group, image B: OPC administration group.

### DETAILED DESCRIPTION

The present invention is further described below with reference to specific embodiments which are only used to explain the present invention, but cannot be taken as a limitation to the present invention. It will be understood by those of ordinary skill in the art that various changes, modifications, substitutions and variations can be made to these embodiments without departing from the principles and spirit of the present invention, and the scope of the present invention is defined by the claims and their equivalents. The experimental method of which specific conditions not indicated in the following examples, and are usually conducted and tested according to conventional conditions or according to the conditions suggested by a manufacturer.

### Example 1 Constructs a Lentiviral Structure

### 1. Experimental Materials

Vector backbone pCW57.1 purchased from addgene (cargo number: plasmid-41393);
Gene synthesis company: Anhui General Biotechnology Co. Ltd.

### 2. Construction Method

The lentiviral vectors constructed in the present embodiment are respectively named as TEToff-CXCL-puromycin, TEToff-IL10-T2A-IL27-Zeo, TEToff-IL3-hygroR.

### (1) Construction of TEToff-CXCL-Puromycin Vector

Synthesizing gene CXCL11 by gene synthesis. The sequence of gene CXCL11 is as shown in SEQ ID NO: 1.

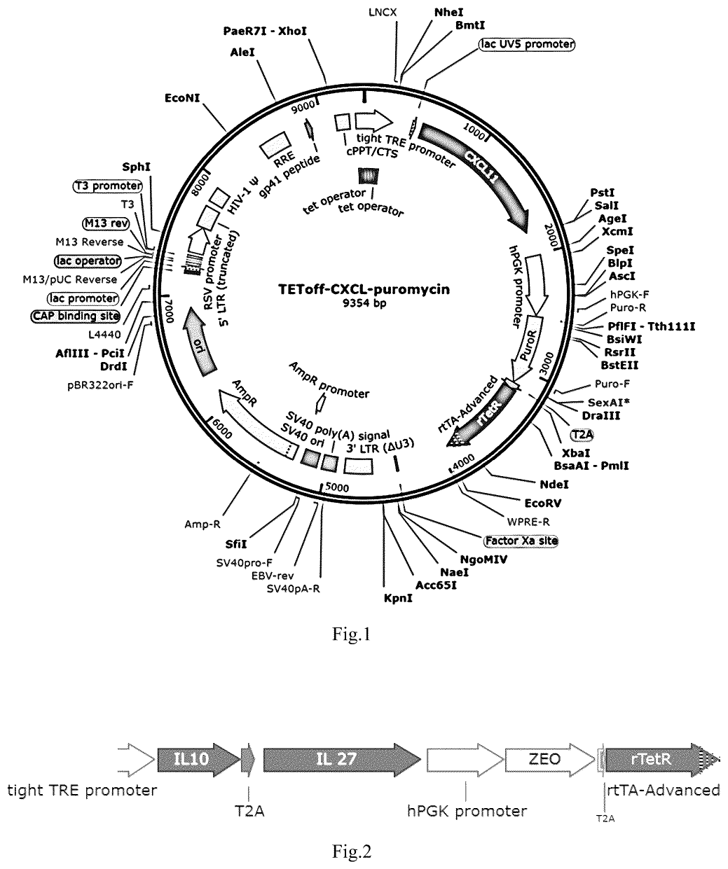

As shown in FIG. 1, the gene CXCL11 was inserted into the segment of Nhe I (3′ end) and Age I (5′ end) of the vector backbone pCW57.1, to obtain the TEToff-CXCL-puromycin;

### (2) Construction of TEToff-IL10-T2A-IL27-Zeo Vector

The structure shown in FIG. 2 was synthesized by gene synthesis, the sequences of core structural gene IL-10, T2A, IL-27, hPGK promoter, Zeocin(ZEO), rtTA-Advanced(rTetR) are shown in SEQ ID NO:2-SEQ ID NO:7.

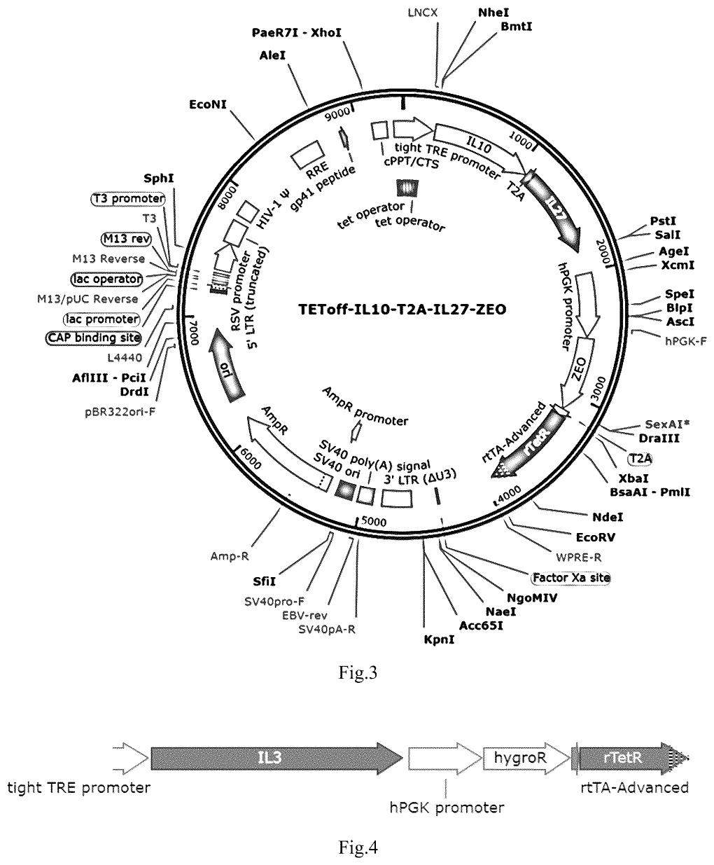

As shown in FIG. 3, the gene-synthesized IL10-T2A-IL27-Zeo was inserted into the segment of Nhe I (3′ end) and EcoRV(5′ end) of the vector backbone pCW57.1, to obtain the TEToff-IL10-T2A-IL27-Zeo;

### (3) Construction of TEToff-IL3-hygroR Vector

Structure shown in FIG. 4 was synthesized by gene synthesis, sequences of the core structural gene IL-3, Hygromycin(hygroR) are shown in SEQ ID NO: 8, SEQ ID NO: 9.

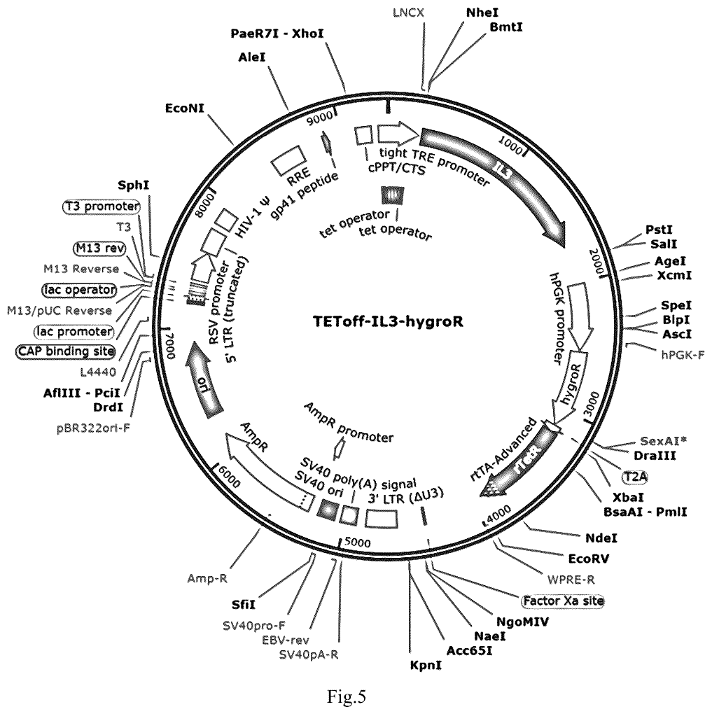

As shown in FIG. 5, the gene-synthesized IL3-hygroR was inserted into the segment of Nhe I (3′ end) and EcoRV(5′ end) of the vector backbone pCW57.1, to obtain the TEToff-IL3-hygroR.

### Example 2 Transfection and Selection of iPSC Strains

### 1. Experimental Materials

The iPSCs used in this embodiment were derived from Beijing Allife Medical Science and Technology Co., Ltd. The lentiviral vector used in this embodiment was the lentiviral vector constructed in Example 1, and other experimental materials used in this embodiment are shown in Table 1.

TABLE 1
Experimental Materials

| Name | Manufacturer | Cargo number |
|---|---|---|
| E8 Basal Medium | STEMCELL | 5991 |
| E8 25X Supplement | STEMCELL | 5992 |
| DMEM/F-12 with HEPES | GIBCO | 11330032 |
| DMEM, High Glucose, | GIBCO | 10566016 |
| matrigel | Corning | 354277 |
| accutase | promocell | C-41310 |
| Y-27632(Rocki) | abcam | ab120129 |
| PEI | POLYSCIENCES, | 23966 |
| polybrene | yeasen | 40804ES76 |
| pan fetal bovine serum | PAN SERATECH | ST3033-02 |
| pMD2.G | addgene | #12259 |
| pCMV-VSVG | addgene | #8454 |
| pRSV-Rev | addgene | #12253 |
| Lenti-XTM Concentrator | clonetech | 631232 |
| puromycin | yeasen | 60210ES25 |
| hygromycin | yeasen | 60224ES03 |
| Zeocin ™ selection reagent | GIBCO | R25001 |

### 2. Cell Culture

(1) The passage was initiated when the cells amplified to cell confluency of 75%-85%. Taking T25 culture dish as an example, sucking away the old culture medium, washing twice with DPBS of room temperature, then adding 3 mL 37° C. preheated Accutase, placing in a cell incubator of 37° C., 5% CO₂ for 5 min, and observing gaps emerging between individual cells under a microscope;
(2) Discarding Accutase, adding 3 mL of TeSR-E8 complete medium to terminate digestion, transferring to a 15 mL centrifuge tube, and centrifuging at 1000 rpm for 5 min at room temperature;
(3) Discarding the supernatant, gently blowing the cells with 1 mL TeSR-E8 medium preheated at 37° C. and added with 10 μM Rocki, and then resuspending. Plating after counting, taking the 6-well plate as an example, the cell suspension of each well was 2 mL, and the plating density was 3×10⁴/well.

### 3. Cell Transfection

Introducing the lentiviral vector TEToff-CXCL-puromycin, TEToff-IL10-T2A-IL27-Zeo, TEToff-IL3-hygroR constructed in Example 1 into iPSCs, selecting iPSCs obtained by transfection, culturing monoclonal iPSCs acquired by selection.

### 3.1 Lentivirus Package

(1) Cell inoculation: 10 cm culture dish was inoculated with 1.5×10⁷ 293T cells. Adding 10 mL of DMEM, High Glucose, GlutaMAX™ medium which containing FBS of 10%, culturing in an incubator of 37° C./5% CO₂ overnight, and transfecting after 16-24 h;
(2) Cell transfection: preparing for transfection when the confluency of cell growth reached 80%-90%. The transfection system is shown in Table 2; the solution B was added dropwise into the solution A while being shaken up, standing for 15 min at room temperature of 22-26° C., being added dropwise into the culture dish, gently shaken up, and cultured overnight at 37° C./5% CO₂;

TABLE 2
Transfection System

| Solution A | | | Solution B | | |
|---|---|---|---|---|---|
| TEToff-CXCL- | 6.65 | μg | PEI | 45 | μg |
| puromycin or TEToff- | | | | | |
| IL10-T2A-IL27-Zeo or | | | | | |
| TEToff-IL3-hygroR | | | | | |
| pMD2.G | 4.3 | μg | serum-free DMEM | 500 | μL |
| pCMV-VSVG | 2.3 | μg | | | |
| pRSV-Rev | 1.68 | μg | | | |
| serum-free DMEM | 500 | μL | | | |

(3) Replacing transfection solution: after 16-18h, removing the culture medium containing the transfection reagent, adding 10 mL of DMEM containing FBS of 10%, and continuing cultivation at the condition of 5% CO₂/37° C. (since this moment, viruses were going to be produced in the cell supernatant);
(4) The first harvest of viruses: after 48h from transfection, the cell supernatant was harvested, transferred to 50 mL of centrifuge tube, centrifuged at 3,000 rpm for 10 min, supernatant was filtered with a filter membrane of 0.45 μm, stored under the condition of 4° C., the cells were added with 10 mL of DMEM containing 10% FBS, and continued to be cultured at 5% CO₂/37° C.
(5) The second harvest of viruses: the cell supernatant was harvested and transferred to a 50 mL centrifuge tube, centrifuged at 3,000 rpm for 10 min, the supernatant was filtered with a filter membrane of 0.45 μm, preserved at 4° C., and the cells were discarded after being treated with 10% disinfectant (the 84 disinfectant);
(6) Virus concentration: respectively filtering the lentivirus groups collected with 0.45 μm filters to remove bacterial contamination, mixing the filtered components with a volume ratio of 3:1, and slightly reversing and uniformly mixing;
(7) Incubating at 4° C. for 30 min or overnight;
(8) Centrifuging at 4° C., 1, 500 g for 45 min, and after centrifugation, white precipitates were seen at the bottom of the tube;
(9) Carefully sucking to remove the supernatant without destroying the white precipitates;
(10) Resuspending the precipitates with an appropriate volume of lentivirus preservation solution, subpackaging the lentivirus, and storing at −80° C.
   3.2 iPSC Lentivirus Infection and Selection
(1) 18-24 hours before the lentivirus transfection, the iPSCs were plated into the 6-well plate for 3×10⁴/well;
(2) On the second day, replacing the original culture medium with culture medium containing 8 μg/mL of fresh TeSR-E8, and adding an appropriate amount of TEToff-CXCL-puromycin virus suspension. Incubating at 37° C.
(3) Continuing cultivation for 24 hours, and replacing the culture medium containing virus with fresh culture medium;
(4) Continuing cultivation. 72-96 hours after transfection, adding 1 μg/mL of puromycin to select positive cells;
(5) Amplifying and culturing positive cells;
(6) Repeating step (1)-(5) using the positive cells described above, in step (2), using TEToff-IL3-hygroR virus solution, and using 50 μg/mL of hygromycin to select positive cells in step (4);
(7) Amplifying and culturing positive cells;
(8) Repeating step (1)-(5) using the positive cells described above, and in step (2), using TEToff-IL10-T2A-IL27-Zeo virus solution, and using 100 μg/mL of zeocin to select positive cells in step (4);
(9) Amplifying and culturing positive cells which named as super-iPSC.

### Example 3: Preparation and Identification of OPCs Derived from iPSCs

### 1. Experimental Materials

The super-iPSCs obtained by construction via transfection and selection in Sample 2 of the present invention. Other experimental materials used in this embodiment are shown in Table 3.

TABLE 3
Experimental Materials

| Common | D-PBS | Life Technologies, cat. no. |
|---|---|---|
| Reagent | | 14190-250 |
| | 5% BSA | sigma, V900933 |
| | 4% Paraformaldehyde | sigma, P6148 |
| | 0.3% Triton X-100 | Sigma-Aldrich, cat. no. |
| | | T9284-100ML |
| Primary | Anti-Olig2 antibody | Abcam, Cat. no. 109186 |
| Antibody | Anti-Nestin antibody | Abcam, Cat. no. 22035 |
| Secondary | Alexa Fluor ® 594- | Abcam, Cat. no. 150108 |
| Antibody | conjugated donkey anti- | |
| | mouse | |
| | Alexa Fluor ® 488- | Abcam, Cat. no. 150073 |
| | conjugated donkey anti- | |
| | rabbit | |
| | Nuclear Markers | Abcam, Cat. no. 104139 |
| | Fluoroshield Mounting | |
| | Medium With DAPI | |
| Components | Non-essential amino acid | Life Technologies, cat. no. |
| of Culture | | 11140-050 |
| Medium | GlutaMAX-I | Life Technologies, cat. no. |
| | | 35050061 |
| | Insulin | Sigma 12643 |
| | SB431542 | medchemexpress, HY-10431 |
| | LDN193189 | medchemexpress, HY-12071 |
| | vitamin A acid | medchemexpress, HY-14649 |
| | β-mercaptoethanol | Gibco, 31350010 |
| | SAG | medchemexpress, HY-12848 |
| | N2 supplement | ThermoFisher, cat. no. |
| | | 17502001 |
| | B27 supplement | ThermoFisher, cat. no. |
| | | 12587010 |

2. Method for Preparing OPCs Derived from iPSCs

### 2.1. Preparing the Following Culture Medium for Use

(1) Neural induction complete medium: 98% DMEM/F-12 medium, 1% non-essential amino acid, 1% GlutaMAX-I, 0.1 mM 2-Mercaptoethanol, 10 μM SB431542, 0.25 μM LDN193189, and 100 μM vitamin A acid, 25 μg/mL insulin;
(2) N2 culture medium: 97% DMEM/F-12 medium, 1% non-essential amino acid, 1% GlutaMAX-I, 0.1 mM 2-Mercaptoethanol, 1% N2 supplement, 1 μM SAG, and 100 μM vitamin A acid;
(3) B27 culture medium: 95% DMEM/F-12 medium, 1% non-essential amino acid, 1% GlutaMAX-I, 0.1 mM 2-Mercaptoethanol, 1% N2 supplement, 2% B27 supplement and 1 μM SAG and 100 μM vitamin A acid, 25 μg/mL insulin;
(4) OPC maturation medium: 95% DMEM/F-12 medium, 1% non-essential amino acid, 1% GlutaMAX-I, 0.1 mM 2-Mercaptoethanol, 1% N2 supplement, 2% B27 supplement, 10 ng/mL PDGF-AA, 10 ng/mL IGF-1, 5 ng/mL HGF, 10 ng/mL NT3, 60 ng/mL T3, 100 ng/mL Biotin, 1 μM cAMP, 25 μg/mL insulin.

### 2.2 Induction of OPCs

(1) From day 0, replacing E8 complete medium with neural induction complete medium for the super-iPSC.

(2) Being placed in a culture incubator of 37° C./5% CO₂;
(3) Then, from day 1 to day 7, replacing the solution every day;
(4) Carefully observing changes in the cell morphology every day;
(5) From day 8, replacing the neural induction complete medium with N2 culture medium;
(6) Placing in a culture incubator of 37° C./5% CO₂;
(7) Then, from day 8 to day 11, replacing the solution every day;
(8) Carefully observing changes in cell morphology every day;
(9) From day 12, replacing the N2 culture medium with the B27 culture medium, and cell cultivation being converted from adherent culture into suspension culture;
(10) On the day 12, pipetting away the old culture medium, and adding the B27 culture medium into each well;
(11) Scraping the cells with sterilized blades, at least 20 times, then respectively, rotating the wells by 900 and 450 and scraping at least 20 times;
(12) Scraping the entire well along the scraping line with a cell scraper to scrape off the cells;
(13) Gently blowing 3-5 times with a 1 mL tip and then transferring 1 well of cells into two wells of a low-attachment 6-well plate; then supplementing B27 culture medium to each well, so that the final volume in each well was 3 mL; placing in culture incubator of 37° C./5% CO₂;
(14) Then from day 12 to day 19, replacing the solution every other day;
(15) Carefully observing changes in the cell morphology every day;
(16) from day 20, replacing the B27 culture medium with OPC maturation medium, and cell cultivation being converted from adherent culture into suspension culture;
(17) On day 20, transferring the spherical aggregation to a 15 mL centrifuge tube with a 1 mL tip, standing for 3 min to sink the spherical aggregation to the bottom of the centrifuge tube, pipetting away ⅔ of the old culture medium, then re-supplementing the OPC maturation medium of the same volume, and then transferring the spherical aggregation back to the original low-attachment 6-well plate again;
(18) From day 20 to day 30, replacing the solution every other day.

### 3. Identification of Prepared OPCs

### 3.1 Preparation of Working Solution

(1) Preparing 10 mL of blocking serum diluent (5% BSA+0.5% Triton X-100+DPBS solution, taking preparing 10 mL as an example. That is, 500 μL of normal 5% BSA and 100 μL of 30% Triton X-100 being added to 9.4 mL of DPBS).
(2) Preparing primary antibody working solution: adding an appropriate titer of the primary antibody to the blocking serum diluent (see the particular value of titer in the primary antibody specification);
(3) Preparing secondary antibody working solution: adding an appropriate titer of the secondary antibody to the blocking serum diluent (see particular value of titer in the secondary antibody specification);
(4) Preparing 90% glycerin: diluted with DPBS.

### 3.2 Immunofluorescence Staining

Washing with DPBS for three times, 3 min each time, fixing with 4% PFA at room temperature for 40 min. Washing with DPBS for three times, 3 min each time. Perforation with 0.5% TritonX-100 for 15 min. Blocking with 5% BSA+0.15% TritonX-100 at room temperature for 1h. Preparing PBST: DPBS+1% BSA+0.15% TritonX-100. Adding primary antibody, at 4 degrees overnight. Recycling the primary antibody solution, washing with PBST for three times, 10 min each time. Adding the secondary antibody, at a ratio of 1:500, at 4 degrees overnight, avoiding light. Washing with PBST three times, 10 min each time. 5 μg/mL DAPI for 2-3 min, avoiding light. Washing with DPBS for 1 time and adding 90% glycerin.

### 4. Experimental Results

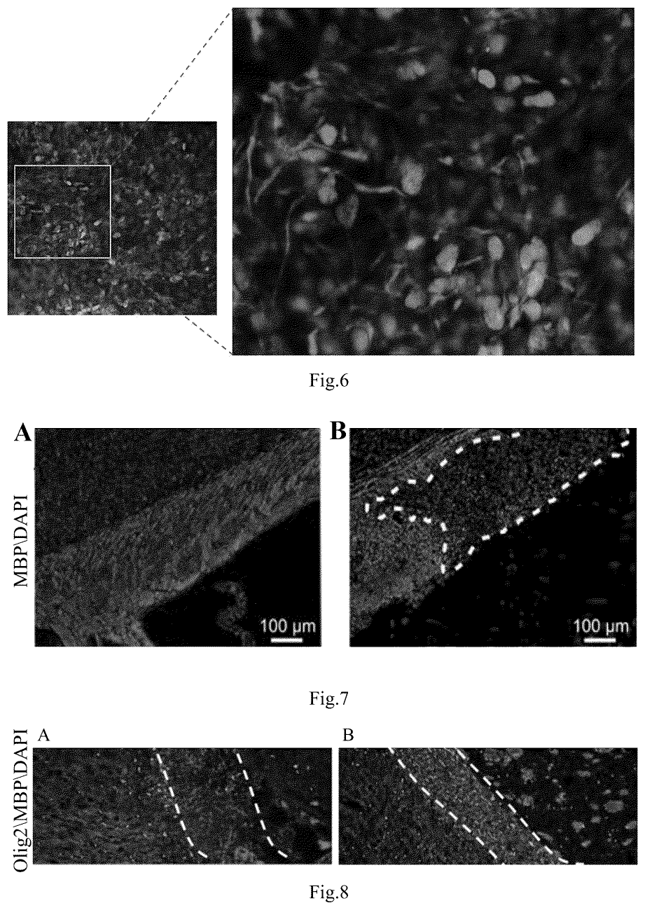

The results shown as FIG. 6, the results show that after the day 30 induction, the Olig2 was expressed in cells, indicating that the present invention successfully prepared the iPSC-derived OPCs, and at the same time, some cells expressed nestin, indicating that some neural stem cells still existed.

### Example 4: Construction of MS Animal Model

### 1. Experimental Materials

C57BL/6 male mice were purchased from Beijing Weitonglihua Experimental Animal Technology Co. Ltd. Bis(cyclohexanone) oxaldihydrazone (CPZ) was purchased from Sigma, Cargo No. C9012

### 2. Experimental Method

8-week-old C57BL/6 male mice were divided into a normal group and an acute demyelination group (CPZ group), and the normal group were fed to normal mouse food every day; the model group were fed with mixed mouse food containing 0.2% CPZ, fed continuously for six weeks; and the demyelination level was determined by the immunohistochemistry of the myelin alkaline protein antibody (MBP).

### 3. Experimental Results

The results shown as FIG. 7A and FIG. 7B, the results show that the control group had a complete myelin sheath and didn't have a demyelinating lesion, and there was demyelination in CPZ group, indicating that the MS animal model was successfully constructed according to the present invention.

### Example 5: Therapeutic Effect of the OPCs Constructed According to the Present Invention on the MS Animal Model

### 1. Experimental Materials

The super-iPSC derived OPCs prepared in Example 3 and the MS animal model constructed in Example 4.

### 2. Experimental Method

After anesthetized, the animal was fixed on the brain stereotaxic apparatus (the tips of ear bars from both sides were inserted into the external auditory canal, so that the head was fixed and kept horizontal, and the anterior fontanel and the posterior fontanel were kept in the same plane as much as possible). Disinfected with Iodine, cut the scalp and subcutaneous tissue along the central line, and peeled the periosteum. The intersection of the coronal suture and the sagittal suture exposed clearly so as to the position of anterior fontanel could be determined and took the anterior fontanel as the 0 point in coordinate. Puncture positioning points were expressed as front-back (AP), midline-outer side (ML), and depth (DV). First, right-side transplantation was performed, and the three-dimensional positioning point was: 0.75 mm back from anterior fontanel, from midline 0.6 mm deviated to right, and 1.1 mm in depth. Using a 5 μL trace syringe to suck 3 μL of physiological saline, and after accurate positioning according to the above-mentioned positioning point, the OPC group were slowly injected with 2 μL of OPC suspension through a syringe, at a speed of 0.2 μL/min; the control group were slowly injected with 2 μL physiological saline through a syringe, at a speed of 0.2 μL/min. Remained for 5 min after injection, slowly took out, pressed by a cotton swab for a moment. After observing of no bleeding nor liquid leaking, performed left-side transplantation, the three-dimensional positioning point was: 0.75 mm back from the anterior fontanel, from midline 0.6 mm deviated to left, 1.1 mm in depth, and the remaining transplantation procedures, doses, etc. are same as that of the transplantation in the right side; after transplantation completed in both sides, the scalp was sewn. 3 months after the surgery, the hippocampal region slices of mouse brain were taken, and the remyelination condition was determined by immunohistochemistry.

### 3. Experimental Results

The results shown as FIG. 8A and FIG. 8B, the results show that there was continuous demyelination in the control group, and myelin was repaired in the OPC group, indicating that the MS related symptoms were obviously alleviated and the myelin was generated in the MS animal model treated with transplantation of the OPCs constructed by the present invention.

The description of the above embodiments is merely used to understand the method and the core idea of the present invention. It should be noted that for those of ordinary skill in the art, several improvements and modifications can be made to the present invention without departing from the principles of the present invention, and these improvements and modifications will also fall within the scope of protection of the claims of the present invention.

1. A construct for genetically modifying an induced pluripotent stem cell to obtain a genetically modified oligodendrocyte progenitor cell, characterized in that the construct comprises nucleotides encoding anti-inflammatory cytokines, and/or nucleotides encoding chemokines;
preferably, the anti-inflammatory cytokines include: IL-10, IL-27, IL-3, IL-2, IL-4, IL-6, IL-10, IL-11, IL-12, IL-13, IL-16, IL-18, IL-22, IL-27, IL-35, IL-37, IL-38, IL-1Ra, TGF-β;
more preferably, the anti-inflammatory cytokines are IL-10, IL-27 or IL-3;
preferably, the chemokines include: CXC chemokine subgroup, CC chemokine subgroup, XC chemokine subgroup, and CX3C chemokine subgroup;
more preferably, the CXC chemokine subgroup includes: CXCL11, CXCL1, CXCL2, CXCL3, CXCL4, CXCL5, CXCL6, CXCL7, CXCL8, CXCL9, CXCL10, CXCL12, CXCL13, CXCL14, CXCL15, CXCL16 and CXCL17;
more preferably, the CC chemokine subgroup includes: CCL1, CCL2, CCL3, CCL4, CCL5, CCL6, CCL7, CCL8, CCL9, CCL 10, CCL 11, CCL12, CCL13, CCL14, CCL15, CCL16, CCL17, CCL18, CCL19, CCL 20, CCL 21, CCL 22, CCL 23, CCL 24, CCL 25, CCL 26, CCL 27, CCL 28;
more preferably, the XC chemokine subgroup includes: XCL1, XCL2;
more preferably, the CX3C chemokine subgroup includes CX3CL1;
most preferably, the chemokines are CXCL11.

2. A vector, characterized in that the vector comprises the construct according to claim 1;
preferably, the vector comprises: the nucleotide encoding IL-10, the nucleotide encoding IL-27, the nucleotide encoding IL-3, and/or the nucleotide encoding CXCL11;
preferably, the vector includes: DNA vector, viral vector;
most preferably, the DNA vector includes: DNA plasmid vector, liposome binding to the DNA plasmid, molecular conjugate binding to the DNA plasmid, and polymer binding to the DNA plasmid;
most preferably, the viral vector includes: adenovirus vector, adeno-associated virus vector, lentiviral vector, retroviral vector, herpes simplex virus vector, baculovirus vector, Sendai virus vector, poxvirus vector, geminivirus vector;
most preferably, the viral vector is lentiviral vector.

3. A genetically modified induced pluripotent stem cell, characterized in that the genetically modified induced pluripotent stem cell comprises the vector according to claim 2, wherein the genetically modified induced pluripotent stem cell expresses IL-10, IL-27, IL-3, and/or CXCL11;
preferably, the genetically modified induced pluripotent stem cell overexpresses IL-10, IL-27, IL-3, and/or CXCL11;
more preferably, the genetically modified induced pluripotent stem cell overexpresses IL-10, IL-27, IL-3, and CXCL11.

4. A genetically modified oligodendrocyte progenitor cell, characterized in that induction differentiation of the genetically modified induced pluripotent stem cell according to claim 3 to obtain the genetically modified oligodendrocyte progenitor cell, wherein the genetically modified oligodendrocyte progenitor cell expresses IL-10, IL-27, IL-3, and/or CXCL11; preferably, the genetically modified oligodendrocyte progenitor cell overexpresses IL-10, IL-27, IL-3, and/or CXCL11;
more preferably, the genetically modified oligodendrocyte progenitor cell overexpresses IL-10, IL-27, IL-3, and CXCL11.

5. A method for preparing the genetically modified induced pluripotent stem cell according to claim 3, wherein the method comprises: delivering the vector to an induced pluripotent stem cell;
preferably, the delivering is achieved by introducing the vector into an induced pluripotent stem cell;
more preferably, the introducing method includes: microinjection, electroporation, DEAE-glucan mediated transfection, TALEN method, ZFN method, non-viral vector mediated transfection, viral vector mediated transfection, transposon technology, CRISPR-Cas9 technology;
most preferably, the non-viral vector mediated transfection includes: liposome transfection, calcium phosphate transfection, chitosan transfection;
most preferably, the viral vector mediated transfection includes: lentivirus infection, retroviral infection, adenovirus infection, adeno-associated virus infection.

6. A method for preparing the genetically modified oligodendrocyte progenitor cell according to claim 4, wherein the method comprises: induction differentiation of the genetically modified induced pluripotent stem cell to obtain the genetically modified oligodendrocyte progenitor cell;
preferably, the induction differentiation comprises the following steps:
(1) first stage induction differentiation: culturing the genetically modified induced pluripotent stem cell with the basal medium added with GlutaMAX-I, 2-Mercaptoethanol, SB431542, LDN193189, vitamin A acid and insulin;
(2) second stage induction differentiation: culturing the cell obtained in step (1) with the basal medium added with GlutaMAX-I, 2-Mercaptoethanol, N2 supplement, SAG, and vitamin A acid;
(3) third stage induction differentiation: culturing the cell obtained in step (2) with the basal medium added with GlutaMAX-I, 2-Mercaptoethanol, N2 supplement, B27 supplement, SAG, vitamin A acid and insulin;
(4) fourth stage induction differentiation: culturing the cell obtained in step (3) with the basal medium added with GlutaMAX-I, 2-Mercaptoethanol, N2 supplement, B27 supplement, PDGF-AA, IGF-1, HGF, NT3, T3, Biotin, cAMP and insulin to obtain the genetically modified oligodendrocyte progenitor cell;
more preferably, the first stage induction differentiation is for 5-9 days in total;
more preferably, the second stage induction differentiation is for 2-6 days in total;
more preferably, the third stage induction differentiation is for 6-10 days in total;
more preferably, the fourth stage induction differentiation is for 9-13 days in total;
most preferably, the first stage induction differentiation is for 7 days in total;
most preferably, the second stage induction differentiation is for 4 days in total;
most preferably, the third stage induction differentiation is for 8 days in total;
most preferably, the fourth stage induction differentiation is for 11 days in total;
more preferably, the culture condition is 37° C., 5% CO₂;
more preferably, the basal medium is DMEM/F-12 medium.

7. An induction differentiation agent for induction differentiation of the genetically modified induced pluripotent stem cells according to claim 3 to obtain the genetically modified oligodendrocyte progenitor cells, characterized in that the induction differentiation agent comprises a first stage induction differentiation agent, a second stage induction differentiation agent, a third stage induction differentiation agent, and a fourth stage induction differentiation agent;
preferably, the first stage induction differentiation agent is consisted of: GlutaMAX-I, 2-Mercaptoethanol, SB431542, LDN193189, vitamin A acid and insulin;
more preferably, the first stage induction differentiation agent further comprises the non-essential amino acid;
most preferably, the content of each component in the first stage induction differentiation agent is respectively: 1% non-essential amino acid, 1% GlutaMAX-I, 0.1 mM 2-Mercaptoethanol, 10 μM SB431542, 0.25 μM LDN193189, 100 μM vitamin A acid, 25 μg/mL insulin;
preferably, the second stage induction differentiation agent is consisted of: GlutaMAX-I, 2-Mercaptoethanol, N2 supplement, SAG, and vitamin A acid;
more preferably, the second stage induction differentiation agent further comprises the non-essential amino acid;
most preferably, the content of each component in the second stage induction differentiation agent is respectively: 1% a non-essential amino acid, 1% GlutaMAX-I, 0.1 mM 2-Mercaptoethanol, 1% N2 supplement, 1 μM SAG, 100 μM vitamin A acid;
preferably, the third stage induction differentiation agent is consisted of: GlutaMAX-I, 2-Mercaptoethanol, N2 supplement, B27 supplement, SAG, vitamin A acid, insulin;
more preferably, the third stage induction differentiation agent further comprises the non-essential amino acid;
most preferably, the content of each component in the third stage induction differentiation agent is respectively: 1% non-essential amino acid, 1% GlutaMAX-I, 0.1 mM 2-Mercaptoethanol, 1% N2 supplement, 2% B27 supplement, 1 M SAG, 100 μM vitamin A acid, 25 μg/mL of insulin;
preferably, the fourth stage induction differentiation agent is consisted of: GlutaMAX-I, 2-Mercaptoethanol, N2 supplement, B27 supplement, PDGF-AA, IGF-1, HGF, NT3, T3, Biotin, cAMP, insulin;
more preferably, the fourth stage induction differentiation agent further comprises the non-essential amino acid;
most preferably, the content of each component in the fourth stage induction differentiation agent is respectively: 1% non-essential amino acid, 1% GlutaMAX-I, 0.1 mM 2-Mercaptoethanol, 1% N2 supplement, 2% B27 supplement, 10 ng/mL PDGF-AA, 10 ng/mL IGF-1, 5 ng/mL HGF, 10 ng/mL NT3, 60 ng/mL T3, 100 ng/mL Biotin, 1 μM cAMP, 25 g/mL insulin.

8. A kit for producing the genetically modified induced pluripotent stem cells and/or the genetically modified oligodendrocyte progenitor cells, wherein the kit comprises:
(I) the constructs and/or
(II) the vectors and/or
(III) induced pluripotent stem cells, and/or
(IV) one or more culture media;
preferably, the culture medium is basal medium added with the induction differentiation agent;
more preferably, the basal medium is DMEM/F-12 medium.

9. A composition, characterized in that the composition comprises the construct and/or the vector, and/or the genetically modified induced pluripotent stem cell, and/or the genetically modified oligodendrocyte progenitor cell;
preferably, the composition includes a pharmaceutical composition;
more preferably, the pharmaceutical composition comprises the genetically modified induced pluripotent stem cell and/or the genetically modified oligodendrocyte progenitor cell;
more preferably, the pharmaceutical composition further comprises pharmaceutically acceptable vectors and/or auxiliary materials;
more preferably, the pharmaceutical composition further comprises one or more therapeutic agents;
most preferably, the therapeutic agent includes: peptide, cell factor, checkpoint inhibitor, mitogen, growth factor, miRNA, dsRNA, mononuclear blood cell, feeder cell, feeder cell component or replacement factor thereof, antibody, chemotherapeutic agent, immunomodulatory drug.

10. Application of the construct according to claim 1, characterized in that the application includes:
(1) application of the construct in preparation of a vector;
(2) application of the construct in preparation of the kit for producing the genetically modified induced pluripotent stem cell and/or the genetically modified oligodendrocyte progenitor cell.

11. Application of the vector according to claim 2, characterized in that the application includes:
(1) application of the vector in preparation of the genetically modified induced pluripotent stem cell and/or the genetically modified oligodendrocyte progenitor cell;
(2) application of the vector in preparation of a drug for treating and/or preventing multiple sclerosis;
(3) application of the vector in preparation of the kit for producing the genetically modified induced pluripotent stem cell and/or the genetically modified oligodendrocyte progenitor cell.

12. Application of the genetically modified induced pluripotent stem cell according to claim 3, characterized in that the application includes:
(1) application of the genetically modified induced pluripotent stem cell in preparation of terminally differentiated cell or precursor cell thereof;
(2) application of the genetically modified induced pluripotent stem cell in preparation of genetically modified oligodendrocyte progenitor cell;
(3) application of the genetically modified induced pluripotent stem cell in preparation of a drug for treating and/or preventing of multiple sclerosis.

13. Application of the genetically modified oligodendrocyte progenitor cell according to claim 4 in preparation of a drug for treating and/or preventing multiple sclerosis.

14. Application of the induction differentiation agent according to claim 7 in preparation of the genetically modified oligodendrocyte progenitor cell.

15. Application of the kit according to claim 8 in production of the genetically modified induced pluripotent stem cell and/or the genetically modified oligodendrocyte progenitor cell.

16. Application of the composition according to claim 9 in preparation of a drug for treating and/or preventing multiple sclerosis.

17. Application of IL-10, IL-27, IL-3 or CXCL11, characterized in that the genetically modified induced pluripotent stem cell according to claim 3 expresses IL-10, IL-27, IL-3, and/or CXCL11, the application includes:
(1) application of IL-10, IL-27, IL-3 or CXCL11 in preparation of the genetically modified induced pluripotent stem cell for treating and/or preventing multiple sclerosis;
(2) application of IL-10, IL-27, IL-3 or CXCL1b in preparation of the genetically modified oligodendrocyte progenitor cell for treating and/or preventing multiple sclerosis;
(3) application of IL-10, IL-27, IL-3 or CXCL1 Iin preparation of a drug for treating and/or preventing multiple sclerosis.

18. Application of IL-10, IL-27, IL-3 or CXCL11, characterized in that the genetically modified oligodendrocyte progenitor cell according to claim 4 expresses IL-10, IL-27, IL-3, and/or CXCL11, the application includes:
(1) application of IL-10, IL-27, IL-3 or CXCL11 in preparation of the genetically modified induced pluripotent stem cell for treating and/or preventing multiple sclerosis;
(2) application of IL-10, IL-27, IL-3 or CXCL11 in preparation of the genetically modified oligodendrocyte progenitor cell for treating and/or preventing multiple sclerosis;
(3) application of IL-10, IL-27, IL-3 or CXCL11 in preparation of a drug for treating and/or preventing multiple sclerosis.


SEQUENCE LISTING (9 sequences)

SEQ ID NO: 1 - DNA, 321 bp, synthetic construct
  source 1..321: mol_type = other DNA
atgagtgtgaagggcatggctatagccttggctgtgatattgtgtgctacagttgttcaaggcttccccatgttcaaaagaggacgctgtctttgcataggccctggggtaaaagcagtgaaagtggcagatattgagaaagcctccataatgtacccaagtaacaactgtgacaaaatagaagtgattattaccctgaaagaaaataaaggacaacgatgcctaaatcccaaatcgaagcaagcaaggcttataatcaaattgaaagaaagaatttttaaaaatatcaaaacatatgaagtcctggaaaagagcatctga

SEQ ID NO: 2 - DNA, 534 bp, synthetic construct
  source 1..534: mol_type = other DNA
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaac

SEQ ID NO: 3 - DNA, 54 bp, synthetic construct
  source 1..54: mol_type = other DNA
gaaggtagaggttctctcctcacttgtggtgatgttgaagaaaaccctggtcca

SEQ ID NO: 4 - DNA, 732 bp, synthetic construct
  source 1..732: mol_type = other DNA
atgggccagacggcaggcgaccttggctggcggctcagcctgttgctgcttcccttgctcctggttcaagctggtgtctggggattcccaaggcccccagggaggccccagctgagcctgcaggagctgcggagggagttcacagtcagcctgcatctcgccaggaagctgctctccgaggttcggggccaggcccaccgctttgcggaatctcacctgccaggagtgaacctgtacctcctgcccctgggagagcagctccctgatgtttccctgaccttccaggcctggcgccgcctctctgacccggagcgtctctgcttcatctccaccacgcttcagcccttccatgccctgctgggagggctggggacccagggccgctggaccaacatggagaggatgcagctgtgggccatgaggctggacctccgcgatctgcagcggcacctccgcttccaggtgctggctgcaggattcaacctcccggaggaggaggaggaggaagaggaggaggaggaggaggagaggaaggggctgctcccaggggcactgggcagcgccttacagggcccggcccaggtgtcctggccccagctcctctccacctaccgcctgctgcactccttggagctcgtcttatctcgggccgtgcgggagttgctgctgctgtccaaggctgggcactcagtctggcccttggggttcccaacattgagcccccagccctga

SEQ ID NO: 5 - DNA, 511 bp, synthetic construct
  source 1..511: mol_type = other DNA
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 6 - DNA, 375 bp, synthetic construct
  source 1..375: mol_type = other DNA
atggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactga

SEQ ID NO: 7 - DNA, 747 bp, synthetic construct
  source 1..747: mol_type = other DNA
atgtctagactggacaagagcaaagtcataaacggagctctggaattactcaatggtgtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaa

SEQ ID NO: 8 - DNA, 459 bp, synthetic construct
  source 1..459: mol_type = other DNA
atgagccgcctgcccgtcctgctcctgctccaactcctggtccgccccggactccaagctcccatgacccagacaacgcccttgaagacaagctgggttaactgctctaacatgatcgatgaaattataacacacttaaagcagccacctttgcctttgctggacttcaacaacctcaatggggaagaccaagacattctgatggaaaataaccttcgaaggccaaacctggaggcattcaacagggctgtcaagagtttacagaacgcatcagcaattgagagcattcttaaaaatctcctgccatgtctgcccctggccacggccgcacccacgcgacatccaatccatatcaaggacggtgactggaatgaattccggaggaaactgacgttctatctgaaaacccttgagaatgcgcaggctcaacagacgactttgagcctcgcgatcttttga

SEQ ID NO: 9 - DNA, 1026 bp, synthetic construct
  source 1..1026: mol_type = other DNA
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatag